SEQUENCE LISTING (16 sequences)

SEQ ID NO: 1 - protein, 16 aa, Artificial Sequence
  R9
GGGRRRRRRRRRLLLL

SEQ ID NO: 2 - protein, 16 aa, Artificial Sequence
  DPV3
RKKRRRESRKKRRRES

SEQ ID NO: 3 - protein, 23 aa, Artificial Sequence
  HPV33L2-455/467
SYFILRRRRKRFPYFFTDVRVAA

SEQ ID NO: 4 - protein, 26 aa, Artificial Sequence
  INV5
AEKVDPVKLNLTLSAAAEALTGLGDK

SEQ ID NO: 5 - protein, 16 aa, Artificial Sequence
  pAnt
RQIKIWFQNRRMKWKK

SEQ ID NO: 6 - protein, 12 aa, Artificial Sequence
  Tat
YGRKKKRRQRRR

SEQ ID NO: 7 - protein, 11 aa, Artificial Sequence
  HIV-Tat
YGRKKRRQRRR

SEQ ID NO: 8 - protein, 17 aa, Artificial Sequence
  Buforin II
RAGLQFPVGRVHRLLRK

SEQ ID NO: 9 - protein, 21 aa, Artificial Sequence
  Transportan
AGYLLGKINLKALAALAKKIL

SEQ ID NO: 10 - protein, 20 aa, Artificial Sequence
  MAP
KLALKLALKALKAALKLAGC

SEQ ID NO: 11 - protein, 16 aa, Artificial Sequence
  KFGF
AAVALLPAVLLALLAP

SEQ ID NO: 12 - protein, 18 aa, Artificial Sequence
  pVEC
LLIILRRRIRKQAHAHSK

SEQ ID NO: 13 - protein, 18 aa, Artificial Sequence
  SynB1
RGGRLSYSRRRFSTSTGR

SEQ ID NO: 14 - protein, 21 aa, Artificial Sequence
  Pep-1
KETWWETWWTEWSQPKKKRKV

SEQ ID NO: 15 - protein, 15 aa, Artificial Sequence
  Pep-7
SDLWEMMMVSLACQY

SEQ ID NO: 16 - protein, 12 aa, Artificial Sequence
  HN-1
TSPLNIHNGQKL

(19) United States
(12) Patent Application Publication
(10) Pub. No.: US 2020/0399647 A1
(43) Pub. Date: Dec. 24, 2020

(54) GENOME EDITING BY POLLEN-MEDIATED TRANSFORMATION

(71) Applicant: UNIVERSITY OF FLORIDA RESEARCH FOUNDATION, Gainesville, FL (US)

(72) Inventors: Marcio Fernando RESENDE, Jr., Gainesville, FL (US); Kelly Mayrink BALMANT, Gainesville, FL (US); Kristen LEACH, Gainesville, FL (US); Matias KIRST, Gainesville, FL (US); Christopher DERVINIS, Gainesville, FL (US)

(21) Appl. No.: 16/769,829

(22) PCT Filed: Dec. 4, 2018
(86) PCT No.: PCT/US18/63746

§ 371 Date: Jun. 4, 2020

Related U.S. Application Data
(60) Provisional application No. 62/594,108, filed on Dec. 4, 2017.

(51) Int. Cl. C12N 15/82

(52) U.S. Cl. CPC C12N 15/8213; C12N 2800/80; C12N 2310/20

(57) ABSTRACT

Disclosed herein are methods of genome-editing transformation that delivers to the cell the RNA-guided endonucleases as well as the required guide RNA, without the need of tissue culture to regenerate the plant. Thus, methods as described herein enable any plant species to have its genome edited in a process that is simple and efficient. This process utilizes genome-editing pollen cells, and delivering the required components for the editing by combining them with cell-penetrating peptides.

### CROSS-REFERENCE TO RELATED APPLICATION

This application claims priority to U.S. Provisional Application entitled “GENOME EDITING BY POLLEN-MEDIATED TRANSFORMATION,” having Ser. No. 62/594,108, filed on Dec. 4, 2017, which is entirely incorporated herein by reference.

### SEQUENCE LISTING

The Sequence Listing for this application is labeled “222110-2570 sequence listing_ST25.txt” which was created on Nov. 30, 2018 and is 8 KB. The entire content of the sequence listing is incorporated herein by reference in its entirety.

### BACKGROUND

Genetic engineering has had a long and successful history in plant biology and crop breeding. Transgenic tools have been extensively used in model species to discover several genetic mechanisms, and have been extensively used in crops to create elite materials, exemplified by the development of virus-resistant papaya and insect resistant maize plants, among many others. More recently, gene editing tools that make use of RNA-guided endonuclease (e.g. CRISPR/Cas9) were developed to increase the precision of the genetic transformation process. Genome-editing methods not only allow the opportunity to more precisely edit the genome, it also allows the genome to be edited without incorporating foreign DNA-sequences, thus creating plants that are edited but non-transgenic.

Current methods for stable genetic engineering and gene editing rely on the delivery of the molecular machinery required to modify the host genome into the nucleus. The delivery is typically mediated by Agrobacterium tumefaciens or biolistic particle delivery system, also known as gene gun. In almost all of the stable transformation methods, the regeneration of the transformed plant requires the use of tissue culture. This step is complex, laborious, and long. More importantly, plant regeneration protocols are genotype specific and many species are recalcitrant to regeneration. This represents a bottleneck in the expansion of genome-editing into non-model plants. Plants species for which there is no established transformation protocols cannot be subjected to genome-editing. Moreover, elite materials in plants with transformation protocols may not be transformable due to the genotype-specific nature of these regeneration protocols.

### SUMMARY

Disclosed herein are methods of genome-editing transformation that can deliver to the cell the RNA-guided endonucleases as well as the required guide RNA (gRNA), without the need of tissue culture to regenerate the plant. Thus, it enables any plant species to have its genome edited in a process that is simple and efficient. This process relies on genome-editing pollen cells, and delivering the required components for the editing by combining them with cell-penetrating peptides (CPPs).

Therefore, disclosed are methods for producing a genome-edited, non-transgenic plant using a pollen-mediated transformation that does not require tissue culture and plant regeneration.

The disclosed methods involve cell penetrating peptides (CPPs) that can be used to deliver large molecules such as gRNA and RNA-guided endonuclease through the plant cell wall to inside the cell.

The disclosed methods involve maintaining viable pollen while delivering the gene editing machinery to the cell. The viable pollen cells are used to pollinate a new plant, which upon fertilization will produce seeds with edited genomes

The details of one or more embodiments of the present disclosure are set forth in the accompanying drawings and the description below. Other features, objects, and advantages of the present disclosure will be apparent from the description and drawings, and from the claims.

Described herein are methods for modifying the genomic material in a plant. Methods as described herein can comprise: (a) providing a RNA-guided endonuclease and cell penetrating peptide (CPP) conjugate; (b) providing one or more guide RNA (gRNA) and CPP conjugates; (c) incubating mature pollen grains from a donor plant in a solution comprising the RNA-guided endonuclease and CPP conjugate and one or more guide RNA and CPP conjugates to form treated pollen grains; and (d) pollinating a maternal plant with the treated pollen grains to produce a plant with modified genomic material.

The RNA-guided endonuclease of methods as described herein can be a Cas9, a Cpf1, a C2c1, ora C2c2. CPPs as described herein can comprise one of SEQ ID NOs. 1-16. CPPs as described herein can comprise a modified variant of one of SEQ ID NOs. 1-16. The gRNA according to methods as described herein can comprise crRNA and a tracrRNA, or a chimeric cr/tracrRNA hybrid.

The donor plant according to methods as described herein can be a monocotyledonous plant, food plant, or dicotyledonous plant. The donor plant according to methods as described herein can be a wheat, maize, rice, orchid, onion, aloe, true lily, grass, Setaria, woody shrub, tree, palm tree, bamboo, pineapple, sugar cane, tomato, cassava, soybean, tobacco, potato, Arabidopsis, rose, pansy, sunflower, grape, strawberry, squash, bean, pea, or peanut. The donor plant according to methods as described herein can be tobacco or maize.

The RNA-guided endonuclease and CPP conjugate and one or more gRNA and CPP conjugates according to methods as described herein can be present in amounts effective to modify genomic DNA in the mature pollen grains. The amounts effective can be a ratio of RNA-guided endonuclease and CPP conjugate and one or more gRNA and CPP conjugates of about 1:1.

The solution according to methods as described herein can comprise glucose or sucrose in an amount of about 1% to about 40%. The solution according to methods as described herein can further comprise calcium nitrate in an amount of about 0.01% to about 0.05%. The solution according to methods as described herein can further comprise boric acid in an amount of about 0.005% to about 0.03%. In an embodiment, the solution comprises about 20% sucrose, about 0.03% calcium nitrate, and about 0.01% boric acid. The solution according to methods as described herein can have a pH of about 5.1 to about 7.5.

The incubating can be for about 30 minutes to about 3 hours. The solution can further comprise a donor DNA sequence and CPP conjugate.

Methods as described herein can further comprise, before step (a), incubating a RNA-guided endonuclease and cell penetrating peptide (CPP) for at least 30 minutes to form a RNA-guided endonuclease and CPP conjugate. In an embodiment, about 2 nM to about 2 μM of RNA-guided endonuclease can be incubated with about 5 nM to about 100 nM CPP. About 10 μM of RNA-guided endonuclease can be incubated with about 50 ng CPP.

Methods as described herein can further comprise, before steps (a) or (b), incubating one or more guide RNA (gRNA) and cell penetrating peptide (CPP) for at least 30 minutes to form one or more gRNA and CPP conjugates. In an embodiment, about 1 ng to about 3 μg of one or more guide RNA (gRNA) can be incubated with about 2 nM to about 2 μM CPP. In an embodiment, about 1 μg of one or more guide RNA (gRNA) can be incubated with about 50 ng CPP.

Methods as described herein can further comprise electroporating, sonicating, or both, the mature pollen grains in solution during step (c), after step (c), or both. Methods as described herein can further comprise storing the treated pollen after step (c). Methods as described herein can further comprise screening the treated pollen for double strand breaks after step (c). Methods as described herein can further comprise screening the genotype of the plant with modified genomic material, phenotype of the plant with modified genomic material, or both, after step (d).

Also described herein are kits. Kits as described herein can comprise: one or more mature pollen grains; a RNA-guided endonuclease; one or more gRNA; and a CPP. The RNA-guided endonuclease of kits as described herein can be a Cas9, a Cpf1, a C2c1, or a C2c2. The CPP of kits as described herein can comprise one of SEQ ID NOs. 1-16. The CPP of kits as described herein can comprise a modified variant of one of SEQ ID NOs. 1-16. The one or more gRNA of kits as described herein can comprise crRNA and a tracrRNA, or a chimeric cr/tracrRNA hybrid.

The one or more mature pollen grains of kits as described herein can be from a monocotyledonous plant, food plant, or dicotyledonous plant. The one or more mature pollen grains of kits as described herein can be from a wheat, maize, rice, orchid, onion, aloe, true lily, grass, Setaria, woody shrub, tree, palm tree, bamboo, pineapple, sugar cane, tomato, cassava, soybean, tobacco, potato, Arabidopsis, rose, pansy, sunflower, grape, strawberry, squash, bean, pea, or peanut. The one or more mature pollen grains of kits as described herein can be tobacco or maize.

Kits as described herein can further comprise a solution, wherein the solution comprises sucrose or glucose in an amount of about 1% to about 40%. The solution of kits as described herein can further comprise calcium nitrate in an amount of about 0.01% to about 0.05%. The solution of kits as described herein can further comprise boric acid in an amount of about 0.005% to about 0.03%. The solution of kits as described herein can comprise about 20% sucrose, about 0.03% calcium nitrate, and about 0.01% boric acid.

### BRIEF DESCRIPTION OF DRAWINGS

FIG. 1 Illustration of an embodiment of the disclosed method for genome editing by pollen-mediated transformation.

FIG. 2 Illustration of a pathway that can be mutated by CPP and Cas9 in tobacco leaves.

FIGS. 3A-3B. Images of tobacco leaves inoculated by CPP and Cas9 for editing phytoene desaturase 3 (PDS3).

FIGS. 4A-4P. Images of pollen germination. FIGS. 4A-4H show images of pollen germination in a sucrose solution 0 minutes after treatment and 30 minutes after treatment following a 30 minute incubation at room temperature (FIGS. 4A-4B, respectively), a one hour incubation at 6° C. (FIGS. 4C-4D, respectively), a two hour incubation at 6° C. (FIGS. 4E-4F, respectively), and fa three hour incubation at 6° C. (FIGS. 4G-4H, respectively). FIGS. 4I-4P show images of pollen germination in a sucrose solution with calcium nitrate and boric acid 0 minutes after treatment and 30 minutes after treatment following a 30 minute incubation at room temperature (FIGS. 4I-4J, respectively), a one hour incubation at 6° C. (FIGS. 4K-4L, respectively), a two hour incubation at 6° C. (FIGS. 4M-4N, respectively), and a three hour incubation at 6° C. (FIGS. 4O-4P, respectively).

FIGS. 5A-5B. Images of successful corn pollination using pollen that was treated in a sucrose solution for 0 and 30 minutes. These figures demonstrate that the pollen is still viable and can create seeds.

FIG. 6. A gel showing that the Cas9 is active in the same solution used for pollen germination.

FIGS. 7A-7D. Bright field and fluorescent images of pollen grains that have been subjected to no treatment (FIGS. 7A-7B) and infused with a GFP/CPP conjugated product (FIGS. 7C-7D) after 3 hours of incubation.

FIGS. 8A-8B. Fluorescent and bright field images of pollen tubes that have been permeated with the GFP/CPP conjugate after allowing the pollen to germinate for 30 minutes in a sucrose solution containing calcium nitrate, boric acid, and the GFP/CPP conjugated product, where arrows indicate corresponding pollen tubes between the bright field and GFP images.

FIG. 9. An illustration of an experimental method to validate genome editing by pollen-mediated transformation using gRNA targeting yl.

FIG. 10. Embodiment of a method of gRNA design and validation.

FIG. 11. Embodiment of a method according to the present disclosure.

FIG. 12. Embodiment of a method according to the present disclosure.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limits of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of genetics, molecular biology, phytology, botany, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is in atmosphere. Standard temperature and pressure are defined as 25° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. Thus, for example, reference to “a support” includes a plurality of supports. In this specification and in the claims that follow, reference will be made to a number of terms that shall be defined to have the following meanings unless a contrary intention is apparent.

The phrases “consisting essentially of” or “consists essentially of” indicate that the claim encompasses embodiments containing the specified materials or steps and those that do not materially affect the basic and novel characteristic(s) of the claim.

The term “about” or “approximately” means within an acceptable error range for the particular value as determined by one of ordinary skill in the art, which will depend in part on how the value is measured or determined, i.e., the limitations of the measurement system and on the parameter being measured. Where particular values are described in the application and claims, unless otherwise stated the term “about” meaning within an acceptable error range for the particular value should be assumed. In the context of compositions containing amounts of ingredients where the term “about” is used, these compositions contain the stated amount of the ingredient with a variation (error range) of 0-10% around the value (X±10%).

In the present disclosure, ranges are stated in shorthand, so as to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. Also, when ranges are used herein, combinations and subcombinations of ranges (e.g., subranges within the disclosed range), and the specific embodiments therein, are included.

### Definitions

Unless otherwise defined, all technical and scientific terms used herein have the same meaning as commonly understood by one of ordinary skill in the art of molecular biology, medicinal chemistry, and/or organic chemistry. Although methods and materials similar or equivalent to those described herein can be used in the practice or testing of the present disclosure, suitable methods and materials are described herein.

As used in the specification and the appended claims, the singular forms “a,” “an,” and “the” may include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, “control” is an alternative subject or sample used in an experiment for comparison purposes and included to minimize or distinguish the effect of variables other than an independent variable.

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins.

As used herein, “nucleic acid” and “polynucleotide” generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide includes DNAs or RNAs as described above that contain one or more modified bases. Thus, DNAs or RNAs comprising unusual bases, such as inosine, or modified bases, such as tritylated bases, to name just two examples, are polynucleotides as the term is used herein. “Polynucleotide” and “nucleic acids” also includes PNAs (peptide nucleic acids), phosphorothioates, and other variants of the phosphate backbone of native nucleic acids. Natural nucleic acids have a phosphate backbone, artificial nucleic acids may contain other types of backbones, but contain the same bases. Thus, DNAs or RNAs with backbones modified for stability or for other reasons are “nucleic acids” or “polynucleotide” as that term is intended herein.

As used herein, “deoxyribonucleic acid (DNA)” and “ribonucleic acid (RNA)” generally refer to any polyribonucleotide or polydeoxribonucleotide, which may be unmodified RNA or DNA or modified RNA or DNA. RNA may be in the form of a tRNA (transfer RNA), snRNA (small nuclear RNA), rRNA (ribosomal RNA), mRNA (messenger RNA), anti-sense RNA, RNAi (RNA interference construct), siRNA (short interfering RNA), or ribozymes.

As used herein, “nucleic acid sequence” and “oligonucleotide” also encompasses a nucleic acid and polynucleotide as defined above.

As used herein, “DNA molecule” includes nucleic acids/polynucleotides that are made of DNA.

As used herein, “wild-type” is the typical form of an organism, variety, strain, gene, protein, or characteristic as it occurs in nature, as distinguished from mutant forms that may result from selective breeding or transformation with a transgene.

As used herein, “identity,” is a relationship between two or more polypeptide or polynucleotide sequences, as determined by comparing the sequences. In the art, “identity” also refers to the degree of sequence relatedness between polypeptide as determined by the match between strings of such sequences. “Identity” can be readily calculated by known methods, including, but not limited to, those described in Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J. Applied Math. 1988, 48: 1073. Preferred methods to determine identity are designed to give the largest match between the sequences tested. Methods to determine identity are codified in publicly available computer programs. The percent identity between two sequences can be determined by using analysis software (e.g., Sequence Analysis Software Package of the Genetics Computer Group, Madison Wis.) that incorporates the Needelman and Wunsch (J. Mol. Biol., 1970, 48: 443-453) algorithm (e.g., NBLAST, and XBLAST). The default parameters are used to determine the identity for the polypeptides or polynucleotides of the present disclosure.

As used herein, “heterologous” refers to compounds, molecules, nucleotide sequences (including genes), and polypeptide sequences (including peptides and proteins) that are different in both activity (function) and sequence or chemical structure. As used herein, “heterologous” can also refer to a gene or gene product that is from a different organism. For example, a human GTP cyclohydrolase or a synthase can be said to be heterologous when expressed in yeast.

As used herein, “homologue” refers to a polypeptide sequence that shares a threshold level of similarity and/or identity as determined by alignment of matching amino acids. Two or more polypeptides determined to be homologues are said to be homologues. Homology is a qualitative term that describes the relationship between polypeptide sequences that is based upon the quantitative similarity.

As used herein, “paralog” refers to a homologue produced via gene duplication of a gene. In other words, paralogs are homologues that result from divergent evolution from a common ancestral gene.

As used herein, “orthologues” refers to homologues produced by speciation followed by divergence of sequence but not activity in separate species. When speciation follows duplication and one homologue sorts with one species and the other copy sorts with the other species, subsequent divergence of the duplicated sequence is associated with one or the other species. Such species specific homologues are referred to herein as orthologues.

As used herein, “xenologs” are homologues resulting from horizontal gene transfer. As used herein, “similarity” is a quantitative term that defines the degree of sequence match between two compared polypeptide sequences.

As used herein, “cell,” “cell line,” and “cell culture” include progeny. It is also understood that all progeny may not be precisely identical in DNA content, due to deliberate or inadvertent mutations. Variant progeny that have the same function or biological property, as screened for in the originally transformed cell, are included.

As used herein, “culturing” refers to maintaining cells under conditions in which they can proliferate and avoid senescence as a group of cells. “Culturing” can also include conditions in which the cells also or alternatively differentiate.

As used herein, “gene” refers to a hereditary unit corresponding to a sequence of DNA that occupies a specific location on a chromosome and that contains the genetic instruction for a characteristic(s) or trait(s) in an organism. As used herein, “synthetic gene” can refer to a recombinant gene comprising one or more coding sequences for a protein of interest, or a synthetically purified protein that is not naturally occurring in its purified state.

As used herein, the term “recombinant” generally refers to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc.). Recombinant also refers to the polypeptide encoded by the recombinant nucleic acid. Non-naturally occurring nucleic acids or polypeptides include nucleic acids and polypeptides modified by man.

As used herein, “cDNA” refers to a DNA sequence that is complementary to a RNA transcript in a cell. It is a man-made molecule. Typically, cDNA is made in vitro by an enzyme called reverse-transcriptase using RNA transcripts as templates.

As used herein, “transformation” or “transformed” refers to the introduction of a nucleic acid (e.g., DNA or RNA) into cells in such a way as to allow expression of the coding portions of the introduced nucleic acid.

As used herein, “stable expression,” “stable incorporation,” “stable transfection” and the like refer to the integration of an exogenous gene into the genome of a host cell, which can allow for long term expression of the exogenous gene.

As used herein “chemical” refers to any molecule, compound, particle, or other substance that can be a substrate for an enzyme in the enzymatic pathway described herein and/or a carboxylesterase enzyme or biochemical pathway. A “chemical” can also be used to refer to a metabolite of a carboxylic ester. As such, “chemical” can refer to nucleic acids, proteins, organic compounds, inorganic compounds, metabolites etc.

As used herein “biologically coupled” refers to the association of or interaction between two or more physically distinct molecules, groups of molecules compounds, organisms, or particles where the association is directly or indirectly mediated between the two or more physically distinct molecules, groups of molecules compounds, organisms or particles via a biologic molecule or compound. This can include direct binding between two biologic molecules and signal transduction pathways.

As used herein, “biological communication” refers to the communication between two or more molecules, compounds, or objects that is mediated by a biologic molecule or biologic interaction.

As used herein, “biologic molecule,” “biomolecule,” and the like refer to any molecule that is present in a living organism and includes without limitation, macromolecules (e.g. proteins, polysaccharides, lipids, and nucleic acids) as well as small molecules (e.g. metabolites and other products produced by a living organism).

As used herein, “regulation” refers to the control of gene or protein expression or function.

As used herein, “native” refers to the endogenous version of a molecule or compound relative to the host cell or population being described.

As used herein, “non-naturally occurring” refers to a non-native version of a molecule or compound or non-native expression or presence of a molecule or compound within a host cell or other composition. This can include where a native molecule or compound is influenced to be expressed or present at a different location within a host, at a non-native period of time within a host, or is otherwise in an altered environment, even when considered within the host. Non-limiting examples include where a protein that is expressed only in the nucleus of a cell is expressed in the cytoplasm of the cell or when a protein that is only normally expressed during the embryonic stage of development is expressed during the adult stage.

As used herein, “encode” refers to the biologic phenomena of transcribing DNA into an RNA that, in some cases, can be translated into a protein product. As such, when a protein is said herein to be encoded by a particular nucleotide sequence, it is to be understood that this refers to this biologic relationship between DNA and protein. It is well established that RNA can be translated into protein based on the triplet code where 3 nucleotides represent an amino acid. This term also includes the idea that DNA can be transcribed into RNA molecules with biologic functions, such as ribozymes and interfering RNA species. As such, when a RNA molecule is said to be encoded by a particular nucleotide sequence it is to be understood that this is referring to the transcriptional relationship between the DNA and RNA species in question. As such “encoding nucleotide” refers to herein as the nucleotide which can give rise through transcription, and in the case of proteins, translation a functional RNA or protein.

As described herein, the phrase “donor plant” refers to the plant to which the genetic modifications according to the present disclosure are performed to produce the desired outcome or phenoype.

A “native gene” or “an endogenous gene” is a gene that is naturally found in a host microorganism; whereas, an “exogenous gene” is a gene introduced into a host microorganism and which was obtained from a microorganism other the host microorganism. Likewise, a “native promoter” or “endogenous promoter” is a promoter that is naturally found in a host microorganism. In contrast, “exogenous promoter” or “heterologous promoter” is a promoter introduced into a host microorganism via a genetic construct and which was obtained from a microorganism different from host microorganism.

As used herein, “coding sequence” or “coding region” refers to the portion[s] of a gene's DNA or RNA that codes for protein.

### Discussion

As disclosed herein, Clustered Regularly Interspersed Short Palindromic Repeats/CRISPR-associated (CRISPR/Cas) system can be used for plant genome engineering by editing pollen genome. The CRISPR/Cas system provides a relatively simple, effective tool for generating modifications in genomic DNA at selected sites. CRISPR/Cas systems can be used to create targeted double-strand or single-strand breaks, and can be used for, without limitation, targeted mutagenesis, gene targeting, gene replacement, targeted deletions, targeted inversions, targeted translocations, targeted insertions, and multiplexed genome modification. This technology can be used to accelerate the rate of functional genetic studies in plants, and to engineer plants with improved characteristics, including enhanced nutritional quality, increased resistance to disease and stress, and heightened production of commercially valuable compounds.

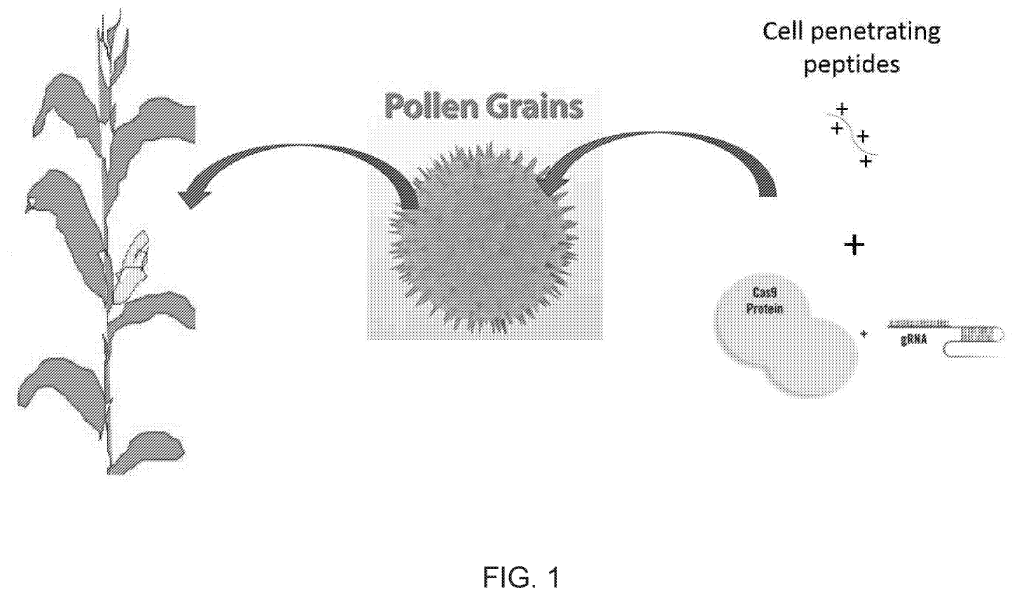

An overview of embodiments of the methods as described herein is shown in FIG. 1. Embodiments of the methods as described herein are believed to be applicable to all species of pollen-producing plants, whether monocotyledonous or dicotyledonous. Of particular interest, of course, are agriculturally important plants, such as field, horticultural, and orchard crops. The procedures for collecting pollen from the anthers of such plants are well established, as are the methods for artificially fertilizing the ovules (eggs) found in the plant ovaries. The collected pollen can be immediately used for DNA editing, or it can be stored under conditions which will substantially preserve its viability and quality. To be effective for gene editing techniques, not only must the pollen viability be maintained, but the pollen tube must maintain its ability to elongate through the style of the flower to reach the ovules. For example, pollen from many plant species can be stored within the range of −70° C. to −20° C., −60° C. to −30° C., and −50° C. to −40° C.

Individual cell penetrating peptides (CPP) can be conjugated with an RNA-guided endonuclease (also referred to herein as “endonuclease”), and one or more guide RNAs (gRNAs). Conjugation as described herein describes primarily the formation of hydrogen bonds between CPPs and other components as described herein (endonuclease, gRNA, donor DNA, and the like). When the genome-editing targets are knock-in or gene insertions, a third conjugation can be performed with a CPP and the donor DNA sequence (which can be a recombinant sequence). All of these conjugations can occur by incubating the CPP with either the endonuclease or the gRNA in a solution with neutral pH at room temperature for at least 30 minutes. In certain aspects, incubation time of CPP with endonuclease and or gRNA can be about 30 minutes to about 2 hours. Incubation times above two hours may not be suitable as CPP residues may be subject to oxidation and therefore loss of CPP efficacy.

In embodiments according to the present disclosure, about 2 nM to about 2 μM of endonuclease can be incubated with CPPs. In embodiments according to the present disclosure an amount of endonuclease that can be incubated with CPPs can be about 50 nM to about 1950 nM, about 100 nM to about 1900 nM, about 150 nM to about 1850 nM, about 200 nM to about 1800 nM, about 250 nM to about 1850 nM, about 300 nM to about 1800 nM, about 350 nM to about 1750 nM, about 400 nM to about 1700 nM, about 450 nM to about 1650 nM, about 500 nM to about 1600 nM, about 550 nM to about 1650 nM, about 600 nM to about 1600 nM, about 650 nM to about 1550 nM, about 700 nM to about 1500 nM, about 750 nM to about 1450 nM, about 800 nM to about 1400 nM, about 850 nM to about 1350 nM, about 900 nM to about 1300 nM, about 950 nM to about 1250 nM, about 1000 nM to about 1200 nM, about 1050 nM to about 1150 nM, or about 1100 nM.

In embodiments according to the present disclosure, about 50 pg to about 2 μg of gRNA can be incubated with CPPs. In embodiments according to the present disclosure, an amount of gRNA that can be incubated with CPPs can be about 0.1 ng to about 1900 ng, about 0.3 ng to about 1700 ng, about 0.5 ng to about 1500 ng, about 0.7 ng to about 1300 ng, about 0.9 ng to about 1100 ng, about 1.1 ng to about 900 ng, about 100 ng to about 800 ng, about 200 ng to about 700 ng, about 300 ng to about 600 ng, or about 400 ng to about 500 ng.

In embodiments according to the present disclosure, about 2 nM to about 50 nM of CPPs are used, about 5 nM to about 45 nM, about 10 nM to about 40 nM, about 15 nM to about 35 nM, about 20 nM to about 30 nM, or about 25 nM.

Mature pollen collected from the donor plant can also be incubated in a solution containing sucrose or glucose at a concentration that maintains viability. The solution may contain other compounds to stabilize the reaction, such as REGULAID® and TWEEN® 20 and induce pollen germination, e.g. boron, calcium, and potassium. The pollen can also be electroporated or sonicated to facilitate the process. In embodiments according to the present disclosure, solutions as described herein can comprise about 1% to about 40% sucrose or glucose, about 5% to about 35% sucrose or glucose, about 10% to about 30% sucrose or glucose, or about 20% sucrose or glucose. In embodiments according to the present disclosure, solutions as described herein can comprise about 0.005% to about 0.03% boric acid, about 0.010% to about 0.025% boric acid, or about 0.015% to about 0.020% boric acid. In embodiments according to the present disclosure, solutions as described herein can comprise about 0.01% to about 0.05% calcium nitrate, about 0.02% to about 0.04% calcium nitrate, or about 0.03% calcium nitrate.

In an embodiment, 10 μM Cas9 and 50 ng CPP can be incubated together, and 1 ug of the gRNA and 50 ng of the CPP can be incubated together.

In a second step, the pollen cells can be treated with the RNA-guided endonuclease conjugated with CPPs and the gRNA conjugated with the CPPs (and optionally the third conjugate of the donor DNA and CPPs). In an embodiment, the RNA-guided endonuclease conjugated with CPPs and the gRNA conjugated with the CPPs can be applied directly to the leaves of a plant of interest that is a plant whose genome is to be edited. Pollen is then incubated in this solution for at least 30 minutes. In certain aspects, pollen is incubated in the solution for about 30 minutes to about 3 hours, about 1 hour to about 2.5 hours, or about 1.5 hours to about 2 hours. In an embodiment, 10 μM Cas9 and 50 ng CPP can be incubated together, 1 μg of the gRNA and 50 ng of the CPP can be incubated together, and the two conjugates can be combined and added to a soluction containing sugar and 10 mg pollen

The treated pollen can then used to pollinate a maternal plant using a brush and conventional techniques to spread the pollen along the silks (the style of the maize flower), or other techniques as known in the art.

Cell penetrating peptides are known in the art and include the TAT transactivation domain of the HIV virus, antennapedia, and transportan which can readily transport molecules and small peptides across the plasma membrane. Non-limiting examples of CPPs, and other internalization molecules, include Polyarginine (e.g., R₉), Antennapedia sequences, TAT, HIV-Tat, Penetratin, Antp-3A (Antp mutant), Buforin II, Transportan, MAP (model amphipathic peptide), K-FGF, Ku70, Prion, pVEC, Pep-1, SynB1, Pep-7, HN-1, BGSC (Bis-Guanidinium-Spermidine-Cholesterol, and BGTC (Bis-Guanidinium-Tren-Cholesterol). Additional cell penetrating peptides are described in U.S. Pat. No. 9,757,473, WO/2014/086835A1, US2017/0057997, and WO/2011/084061, the entireties of each of which are incorporated by reference for these peptides. Finding suitable CPP sequences is within the skill of one of the art, and additional examples of CPPs can be found for example by accessing the CPP 2.0 database (natural or non-natural) on the World Wide Web.

Examples of CPPs and sequences thereof can be found in the Examples below (which can be considered a sequence in order from N-terminal to C-terminal, or a sequence in order from C-terminal to N-terminal). Additionally described herein are modified CPPs. Modified CPPs can comprise a C-terminal modification, an N-terminal modification, or both. In an embodiment, a modification as described herein is the addition of a 4-maleimidobutyrl group to the C-terminal or the N-terminal. Further examples of CPPs as desribed herein are R9 (GGGRRRRRRRRRLLLL-NH2), mR9 (4-maleimidobutyrl-GGGRRRRRRRRRLLLL-NH2), mDVP3 (4-maleimidobutyrl-RKKRRRESRKKRRRES-NH2), mHPV33L2-455/467 (4-maleimidobutyrl-SYFI LRRRRKRFPYFFTDVRVAA-NH2), and ml NV5 (4-maleimidobutyrl-AEKVDPVKLNLTLSAAAEALTGLGDK-NH2). Futher examples of modifications to CPP sequences for a given purpose are known in the art, and such examples are provided in databases such as those curated by commercial organizations such as Genscript and can be found by the skilled artisan for example on the World Wide Web by accessing the Genscript peptide modification site. Further examples can include addition of small peptides from sources such as bovine serum albumin (BSA).

RNA-guided endonuclease for use in the disclosed methods are known in the art, and include naturally occurring DNA binding proteins having nuclease activity, such as Cas9, Cpf1, C2c1, and C2c2 proteins. Cas9 proteins are known to exist in many Type II CRISPR systems including the following as identified in the supplementary information to Makarova et al., Nature Reviews, Microbiology, Vol. 9, June 2011, pp. 467-477: Methanococcus maripaludis C7; Corynebacterium diphtheriae; Corynebacterium efficiens YS-314; Corynebacterium glutamicum ATCC 13032 Kitasato; Corynebacterium glutamicum ATCC 13032 Bielefeld; Corynebacterium glutamicum R; Corynebacterium kroppenstedtii DSM 44385;

Mycobacterium abscessus ATCC 19977; Nocardia farcinica IFM10152; Rhodococcus erythropolis PR4; Rhodococcus jostii RHA1; Rhodococcus opacus B4 uid36573; Acidothermus cellulolyticus 11B; Arthrobacter chlorophenolicus A6; Kribbella flavida DSM 17836 uid43465; Thermomonospora curvata DSM 43183; Bifidobacterium dentium Bd1; Bifidobacterium longum DJO10A; Slackia heliotrinireducens DSM 20476; Persephonella marina EX H1; Bacteroides fragilis NCTC 9434; Capnocytophaga ochracea DSM 7271; Flavobacterium psychrophilum JIP02 86; Akkermansia muciniphila ATCC BAA 835; Roseiflexus castenholzii DSM 13941; Roseiflexus RS1; Synechocystis PCC6803; Elusimicrobium minutum Pei191; uncultured Termite group 1 bacterium phylotype Rs D17; Fibrobacter succinogenes S85; Bacillus cereus ATCC 10987; Listeria innocua; Lactobacillus casei; Lactobacillus rhamnosus GG; Lactobacillus salivarius UCC118; Streptococcus agalactiae A909; Streptococcus agalactiae NEM316; Streptococcus agalactiae 2603; Streptococcus dysgalactiae equisimilis GGS 124; Streptococcus equi zooepidemicus MGCS10565; Streptococcus gallolyticus UCN34 uid46061; Streptococcus gordonii Challis subst CH1; Streptococcus mutans NN2025 uid46353; Streptococcus mutans; Streptococcus pyogenes M1 GAS; Streptococcus pyogenes MGAS5005; Streptococcus pyogenes MGAS2096; Streptococcus pyogenes MGAS9429; Streptococcus pyogenes MGAS10270; Streptococcus pyogenes MGAS6180; Streptococcus pyogenes MGAS315; Streptococcus pyogenes SSI-1; Streptococcus pyogenes MGAS10750; Streptococcus pyogenes NZ131; Streptococcus thermophiles CNRZ1066; Streptococcus thermophiles LMD-9; Streptococcus thermophiles LMG 18311; Clostridium botulinum A3 Loch Maree; Clostridium botulinum B Eklund 17B; Clostridium botulinum Ba4 657; Clostridium botulinum F Langeland; Clostridium cellulolyticum H10; Finegoldia magna ATCC 29328; Eubacterium rectale ATCC 33656; Mycoplasma gallisepticum; Mycoplasma mobile 163K; Mycoplasma penetrans; Mycoplasma synoviae 53; Streptobacillus moniliformis DSM 12112; Bradyrhizobium BTAi1; Nitrobacter hamburgensis X14; Rhodopseudomonas palustris BisB 18; Rhodopseudomonas palustris B is B5; Parvibaculum lavamentivorans DS-1; Dinoroseobacter shibae DFL 12; Gluconacetobacter diazotrophicus Pal 5 FAPERJ; Gluconacetobacter diazotrophicus Pal 5 JGI; Azospirillum B510 uid46085; Rhodospirillum rubrum ATCC 11170; Diaphorobacter TPSY uid29975; Verminephrobacter eiseniae EF01-2; Neisseria meningitides 053442; Neisseria meningitides alpha 14; Neisseria meningitides Z2491; Desulfovibrio salexigens DSM 2638; Campylobacter jejuni doylei 269 97; Campylobacter jejuni 81116; Campylobacter jejuni; Campylobacter lari RM2100; Helicobacter hepaticus; Wolinella succinogenes; Tolumonas auensis DSM 9187; Pseudoalteromonas atlantica T6c; Shewanella pealeana ATCC 700345; Legionella pneumophila Paris; Actinobacillus succinogenes 130Z; Pasteurella multocida; Francisella tularensis novicida U112; Francisella tularensis holarctica; Francisella tularensis FSC 198; Francisella tularensis tularensis; Francisella tularensis WY96-3418; and Treponema denticola ATCC 35405. RNA guided DNA binding proteins also include homologs and orthologs of Cas9, Cpf1, C2c1, and C2c2 proteins that retain the ability of the protein to bind to the DNA, be guided by the RNA and cut the DNA.

According to some aspects, an engineered Cas9 gRNA system is provided which enables RNA-guided genome cutting in a site specific manner in a stem cell, if desired, and modification of the pollen genome by insertion of exogenous donor nucleic acids. The guide RNAs can be complementary to target sites or target loci on the pollen DNA. The guide RNAs can be crRNA-tracrRNA chimeras. The guide RNAs can be introduced to the pollen using CPPs as disclosed herein.

Methods for pollen-mediated gene transformations of plants has been described in U.S. Pat. Nos. 5,049,500, 5,629,183, and CN 102127567, each of which is incorporated by reference for these teachings. These methods involve introducing a foreign DNA into the pollen, e.g by electroporation. However, this results in a foreign DNA in the final plant, which is undesirable for many applications. The disclosed methods instead involves editing the genome of the pollen using Cas9 and a gRNA, which are introduced to the pollen using a cell penetrating peptide (CPP). Briefly, after pollen has been incubated with a CPP, Cas9, and gRNA, the pollen containing the edited DNA can be placed onto a receptive silk, and the pollen will transfer the edited DNA to the ovule (ovum, egg) upon fertilization. After seed set, the resulting seed is examined for the presence of predicted genetic changes via sequencing or visual phyenotypes. DNA transport by pollen may occur within a cultivar or may between cultivars.

Exemplary monocotyledonous plants include, without limitation, wheat, maize, rice, orchids, onion, aloe, true lilies, grasses (e.g., Setaria), woody shrubs and trees (e.g., palms and bamboo), and food plants such as pineapple and sugar cane. Exemplary dicotyledonous plants include, without limitation, tomato, cassava, soybean, tobacco, potato, Arabidopsis, rose, pansy, sunflower, grape, strawberry, squash, bean, pea, and peanut. In an embodiment, the plant, pollen, or both is N. benthamiana. In an embodiment, the plant, pollen, or both is Zea maize.

In some embodiments, the methods described herein can include screening the plant, plant structure, or plant cell to determine if a double-stranded break (DSB) has occurred at or near the sequence targeted by the crRNA and tracrRNA or the cr tracrRNA hybrid. For example, the PCR-digest assay described by Zhang et al. (supra) can be used to determine whether a DSB has occurred. Other useful methods include, without limitation, the T7 assay, the Surveyor assay, and southern blotting (if a restriction enzyme binding sequence is present at or near the predicted cleavage site).

In addition, in some embodiments in which a plant part or plant cell is used, the methods provided herein can include regenerating a plant from the plant part or plant cell. The methods also can include breeding the plant (e.g., the plant into which the nucleic acids were introduced, or the plant obtained after regeneration of the plant part or plant cell used as a starting material) to obtain a genetically desired plant lineage. Methods for regenerating and breeding plants are well established in the art.

Also described herein are kits. Kits as described herein can comprise one or more mature pollen grains; a RNA-guided endonuclease; one or more gRNA; and a CPP. The RNA-guided endonuclease of the kit can be a Cas9, a Cpf1, a C2c1, or a C2c2. The CPP of the kit can comprise one of SEQ ID NOs. 1-16. The CPP of the kit can comprise a modified variant of one of SEQ ID NOs. 1-16. The gRNA of the kit can comprise crRNA and a tracrRNA, or a chimeric cr/tracrRNA hybrid, or other gRNA known to be paired with Cas proteins. The one or more mature pollen grains of the kit can be from a monocotyledonous plant, food plant, or dicotyledonous plant. The one or more mature pollen grains of the kit can be from a wheat, maize, rice, orchid, onion, aloe, true lily, grass, Setaria, woody shrub, tree, palm tree, bamboo, pineapple, sugar cane, tomato, cassava, soybean, tobacco, potato, Arabidopsis, rose, pansy, sunflower, grape, strawberry, squash, bean, pea, or peanut. The one or more mature pollen grains of the kit can be tobacco or maize.

Kits as described herein can further comprise a solution, wherein the solution comprises sucrose or glucose in an amount of about 1% to about 40%, about 5% to about 35%, about 10% to about 30%, or about 20%. The solution can comprise about 20% sucrose, about 0.03% calcium nitrate, and about 0.01% boric acid.

A number of embodiments of the methods for pollen-mediated gene transformation according to the present disclosure have been described. Nevertheless, it will be understood that various modifications may be made without departing from the spirit and scope of the present disclosure. Accordingly, other embodiments are within the scope of the following claims.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

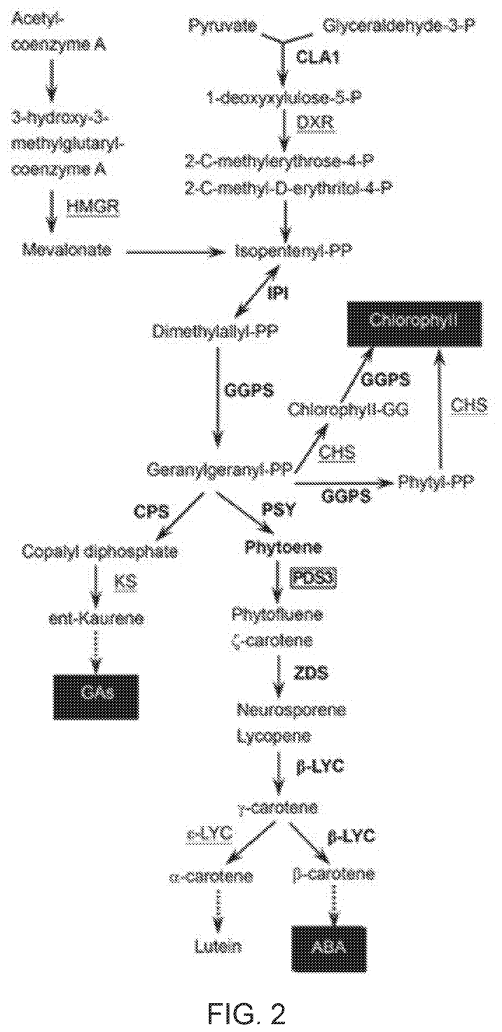

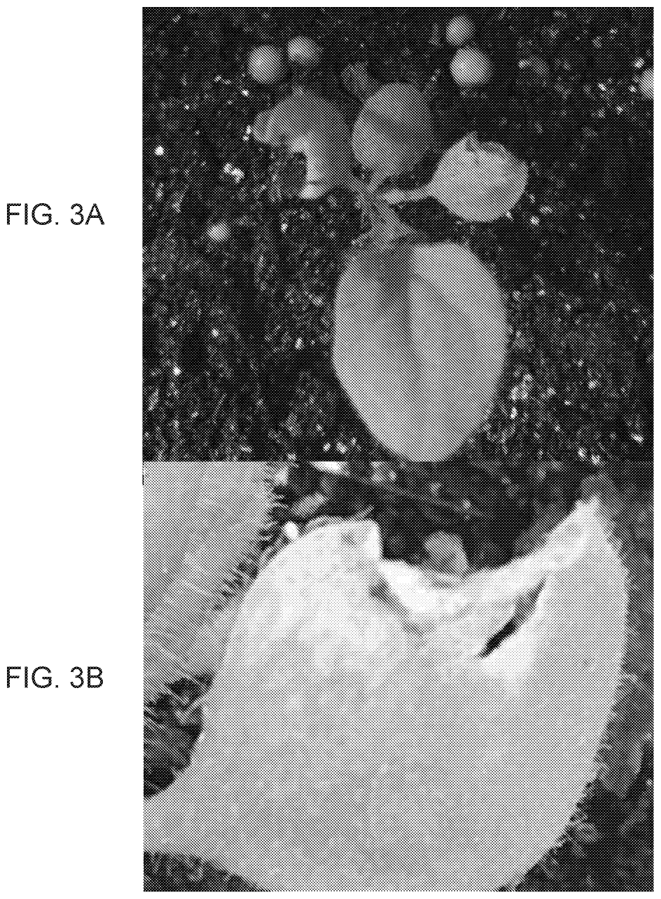

FIG. 2 is an example of pathways of which any protein therein can be modified according to methods as described herein. According to the present example, 10 μM Cas9 and 50 ng CPP were incubated together at room temperature for 2 hours with gentle inversion. In addition, 1 ug of the gRNA and 50 ng of the CPP were incubated together at room temperature for 2 hours with gentle inversion. After the incubation time, the contents of both tubes were combined and infiltrated into N. benthamiana leaves (FIGS. 3A-3B).

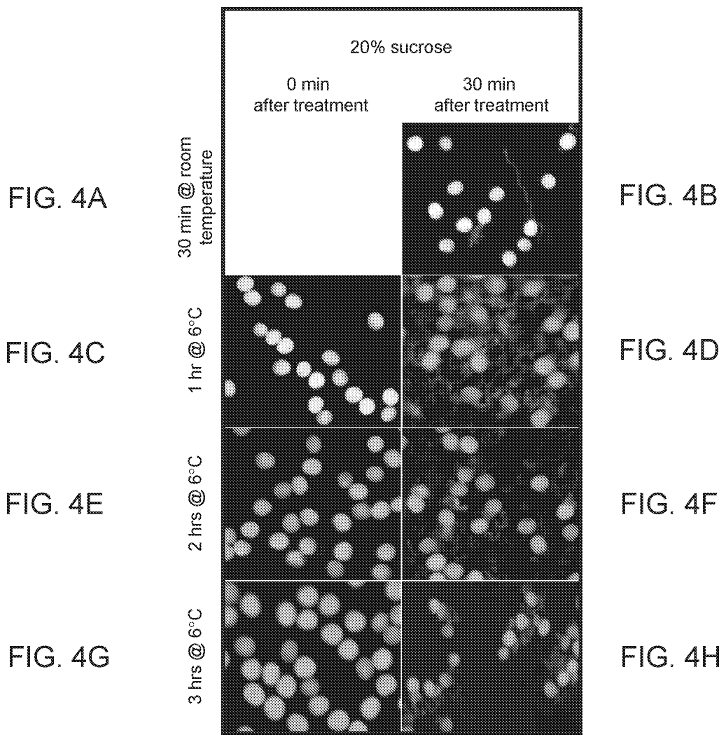

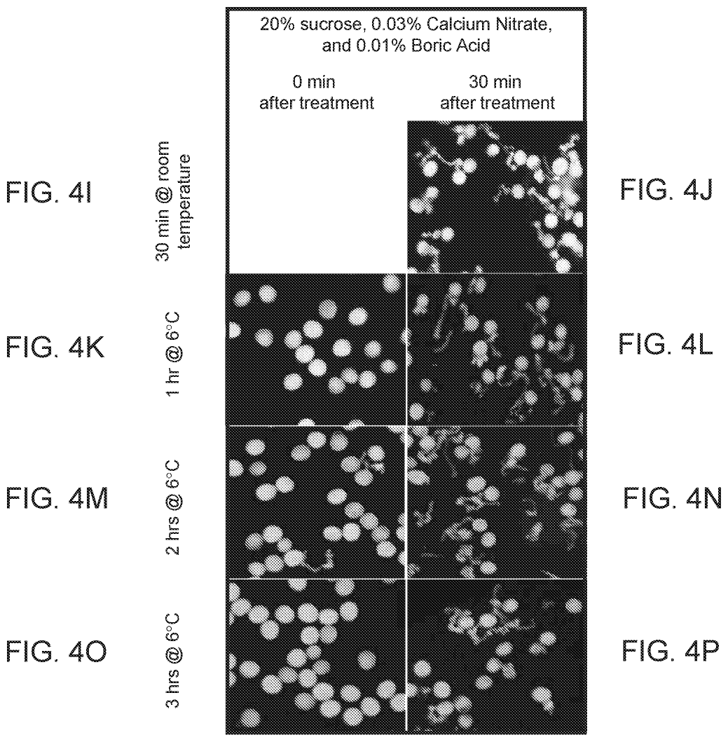

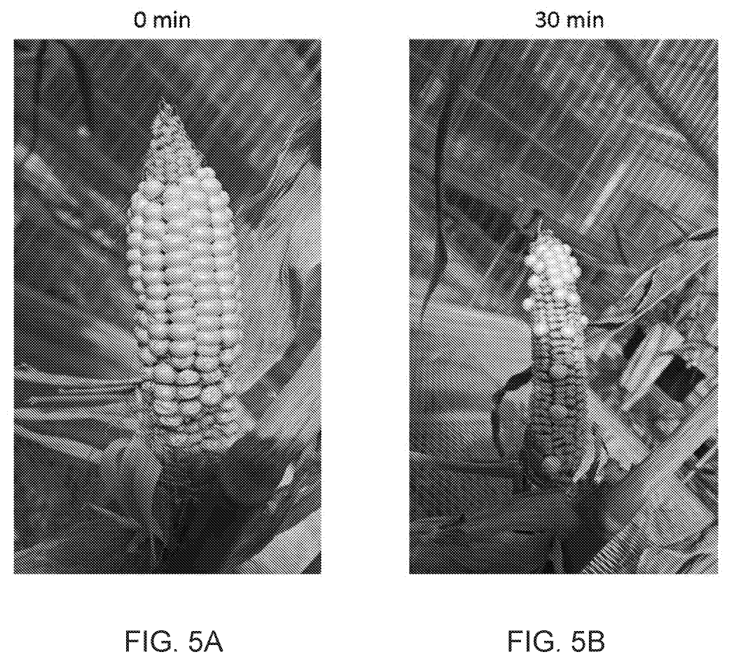

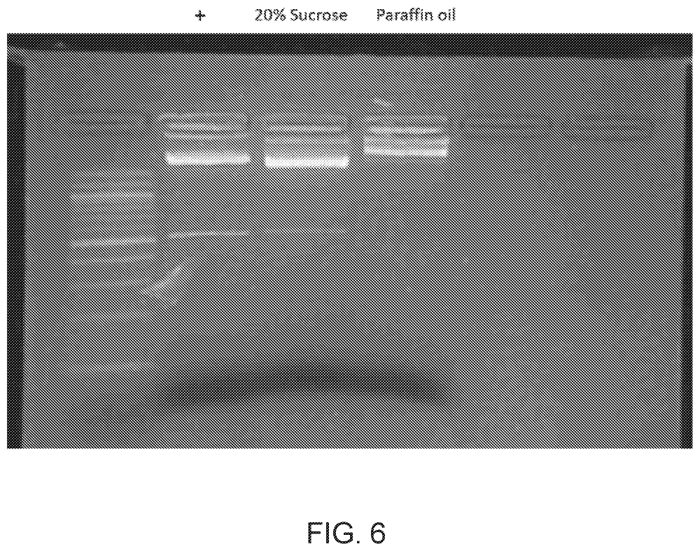

Cas9 and gRNA were also used to recognize and cut a plasmid, incubated for 2 hours in a solution with sucrose (FIG. 6). Pollination was successfully performed by adding 10 mg of Z. maize pollen suspended in 20% sucrose. Pollen germination was observed in the microscope after incubating the pollen below 10° C. for less than three hours in a sucrose solution or a sucrose solution containing boric acid and calcium nitrate (FIGS. 4A-4P). This suspension was allowed to incubate at room temperature for 0 and 30 minutes. In the case of 30 minutes, after that period the suspension was centrifuged at 100×g for 1 minute or allowed to settle. The supernatant was removed and 300 uL of a solution containing 20% sucrose, boric acid and calcium nitrate or light paraffin oil was added to the pollen. Using a #10 camel hair paint brush the pollen was gently applied to silks that were about 20 cm in length. The ear was then bagged to protect it from additional exposure to pollen (FIGS. 5A-5B).

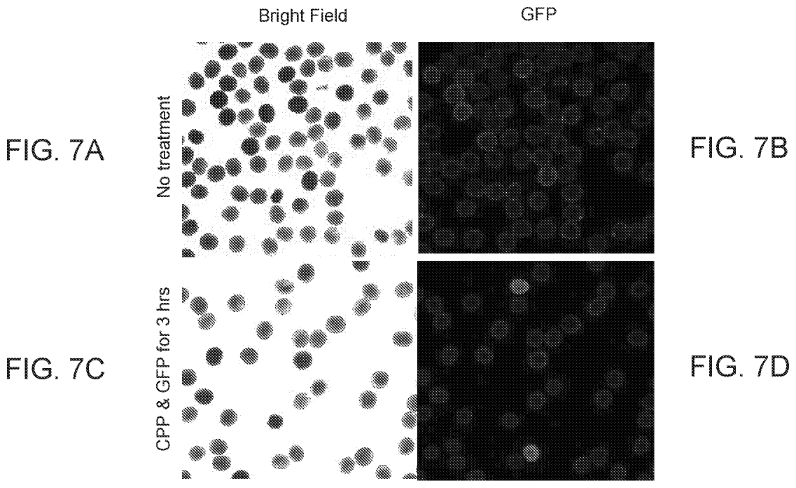

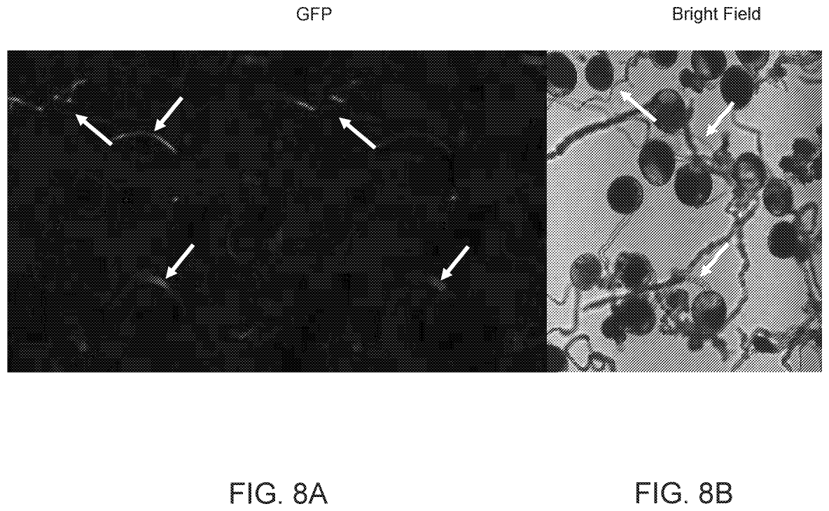

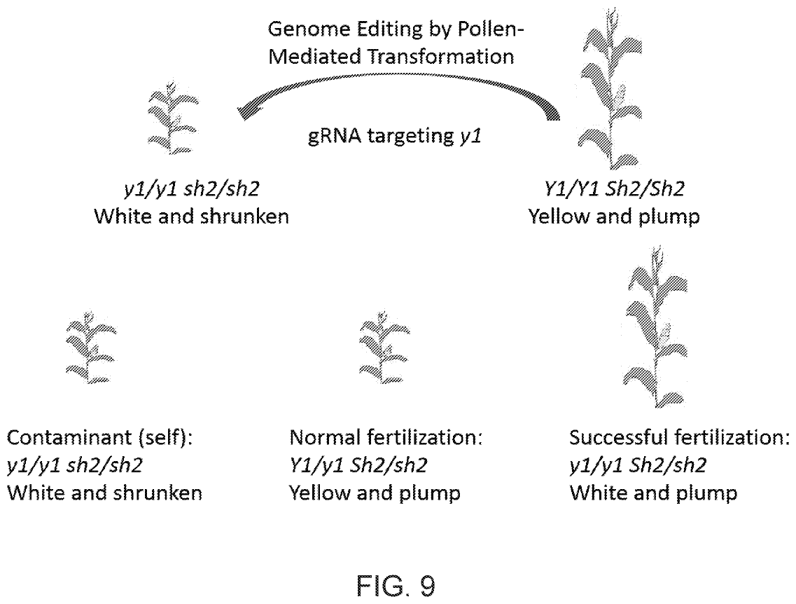

Green fluorescent protein (GFP) and a CPP were incubated together in a sucrose solution at room temperature for an hour to allow for conjugation. The conjugated product was then combined with freshly collected maize pollen (Z. maize), whose viability had been observed under a microscope, allowed to incubate for 3 hours and then visualized to identify pollen grains that had been infused with GFP (FIG. 6). Fresh maize pollen (Z. maize) was also incubated for 30 minutes in a sucrose solution with the addition of boric acid, calcium nitrate and the GFP/CPP conjugated product, then visualized to reveal pollen tubes that had been infused with GFP (FIGS. 7A-7D)

### Example 2

In embodiments according to the present disclosure, a CPP as described herein can comprise a peptide sequence selected from (see Table 1 below): R9 (SEQ ID NO. 1), DPV3 (SEQ ID NO. 2), HPV33L2-455/467 (SEQ ID NO. 3), INV5 (SEQ ID NO. 4), pAnt (SEQ ID NO. 5), Tat (SEQ ID NO. 6), HIV-Tat (SEQ ID NO. 7), Buforin II (SEQ ID NO. 8), Transportan (SEQ ID NO. 9), MAP (SEQ ID NO. 10), KFGF (SEQ ID NO. 11), pVEC (SEQ ID NO. 12), SynB1 (SEQ ID NO. 13), Pep-1 (SEQ ID NO. 14), Pep-7 (SEQ ID NO. 15), and HN-1 (SEQ ID NO. 16).

TABLE 1
Embodiments of sequences of CPP's
according to the present disclosure

| | | SEQ |
|---|---|---|
| Name | Sequence | ID NO: |
| R9 | GGGRRRRRRRRRLLLL | 1 |
| DPV3 | RKKRRRESRKKRRRES | 2 |
| HPV33L2-455/467 | SYFILRRRRKRFPYFFTDVRVAA | 3 |
| INV5 | AEKVDPVKLNLTLSAAAEALTGLGDK | 4 |
| pAnt | RQIKIWFQNRRMKWKK | 5 |
| Tat | YGRKKKRRQRRR | 6 |
| HIV-Tat | YGRKKRRQRRR | 7 |
| Buforin II | RAGLQFPVGRVHRLLRK | 8 |
| Transportan | AGYLLGKINLKALAALAKKIL | 9 |
| MAP | KLALKLALKALKAALKLAGC | 10 |
| KFGF | AAVALLPAVLLALLAP | 11 |
| pVEC | LLIILRRRIRKQAHAHSK | 12 |
| SynB1 | RGGRLSYSRRRFSTSTGR | 13 |
| Pep-1 | KETWWETWWTEWSQPKKKRKV | 14 |
| Pep-7 | SDLWEMMMVSLACQY | 15 |
| HN-1 | TSPLNIHNGQKL | 16 |

CPPs as described herein (such as any of SEQ ID NOs. 1-16) can further be conservatively modified. An example of a conservative modification that can be undertaken is addition of a 4-maleimiodobutyrl group to the CPP peptide sequence, for example on the C-terminus of the peptide. Examples of modified CPPs as used herein include mR9 (4-maleimiodobutyrl-GGGRRRRRRRRRLLLL-NH2), mDPV3 (4-maleimiodobutyrl-RKKRRRESRKKRRRES-NH2), mHPV33L2-455/467 (4-maleimiodobutyrl-SYFI LRRRRKRFPYFFTDVRVAA-NH2), and ml NV5 (4-maleimiodobutyrl-AEKVDPVKLNLTLSAAAEALTGLGDK-NH2).

### Example 3

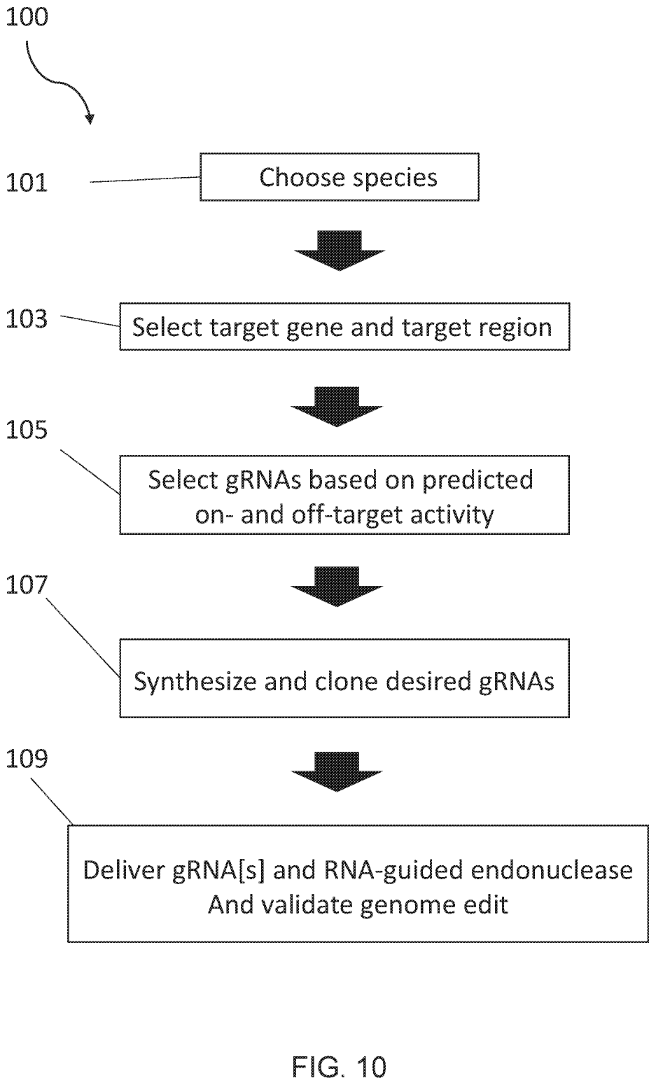

Methods of selecting, desiging, and validating gRNA are known in the art. FIG. 10 is a flow chart of an embodiment of a method to select one or more gRNAs according to methods as known in the art. According to the method 100 of FIG. 10, a desired species is chosen 101 (for example a species of tobacco or maize). One or more genes and target regions of the genome of the species is selected 103 (for example, a gene of the pathway of FIG. 2), and gRNAs are then designed and selected based on predicted on- and off-target activity 105 (can be done with the aid of a variety of software tools). Selected gRNAs can then be syntheized and cloned 107, delivered to the species with a RNA-guided endonuclease and the genome edit validated 109.

### Example 4

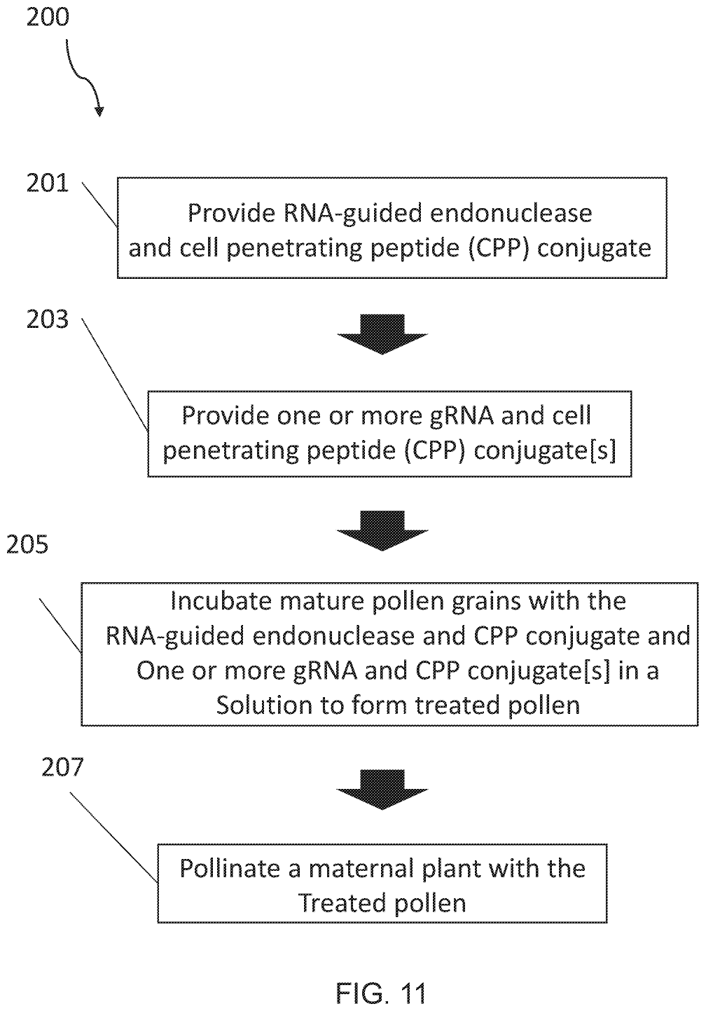

The flow chart of FIG. 11 illustrates an example of an embodiment of a method 200 according to the present disclosure. According to the method 200, a RNA-guided endonuclease and cell penetrating peptide (CPP) conjugate is provided 201, and one or more guide RNA (gRNA) and CPP conjugates are provided 203. Mature pollen grains are then incubated with the RNA-guided endonuclease and CPP conjugate and one or more gRNA and CPP conjugates in a solution to form treated pollen 205. A maternal plant can then be pollinated with the treated pollen 207. Following pollination, the plant can then be monitored for the desire genotypic change by observing phenotype, or by other molecular genetic techniques known in the art (for example restriction cut and polymerase chain reaction, sequence, and the like). Transformation can be aided by electroporating or sonicating (or both) during, after, or both the incubating step 205.

Optionally, the method described herein can include screening the plant, plant structure, or plant cell to determine if a double-stranded break (DSB) has occurred at or near the sequence targeted by the gRNA (crRNA and tracrRNA or the cr tracrRNA hybrid). For example, the PCR-digest assay described by Zhang et al. (supra) can be used to determine whether a DSB has occurred. Other useful methods include, without limitation, the T7 assay, the Surveyor assay, and southern blotting (if a restriction enzyme binding sequence is present at or near the predicted cleavage site).

### Example 5

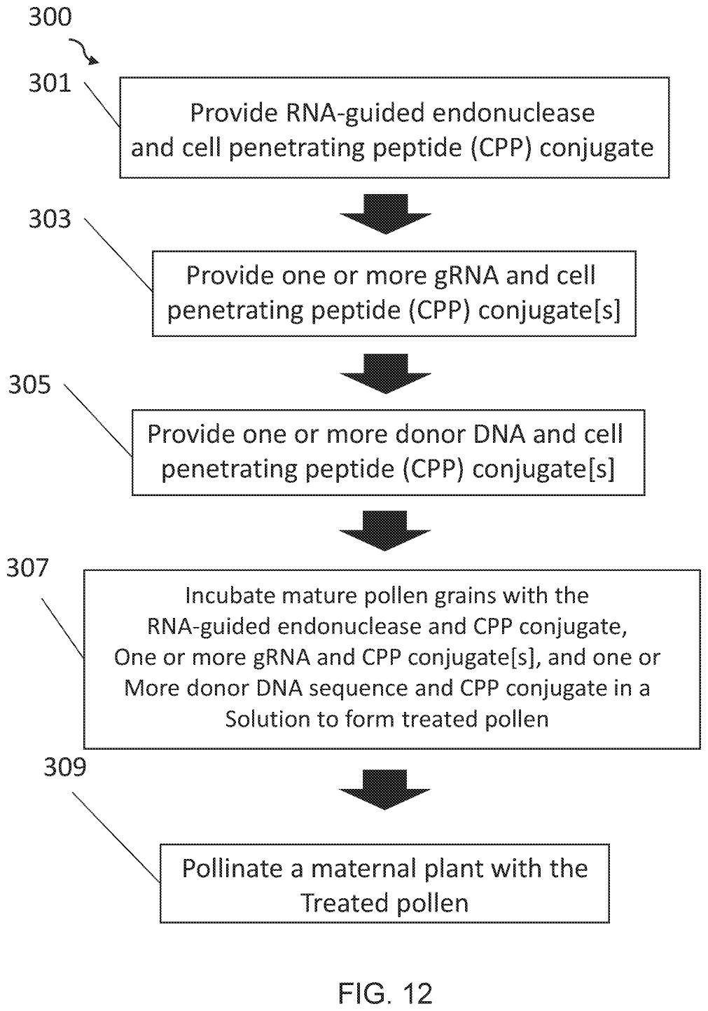

The flow chart of FIG. 12 illustrates an example of an embodiment of a method 300 according to the present disclosure, wherein the desired genome-editing targets are knock-in or gene insertions. According to the method 300, a RNA-guided endonuclease and cell penetrating peptide (CPP) conjugate is provided 301, and one or more guide RNA (gRNA) and CPP conjugates are provided 303. Mature pollen grains are then incubated with the RNA-guided endonuclease and CPP conjugate and one or more gRNA and CPP conjugates in a solution to form treated pollen 307. A maternal plant can then be pollinated with the treated pollen 309. Following pollination, the plant can then be monitored for the desire genotypic change by observing phenotype, or by other molecular genetic techniques known in the art (for example restriction cut and polymerase chain reaction, sequence, and the like). Transformation can be aided by electroporating or sonicating (or both) during, after, or both the incubating step 307.

Optionally, the method described herein can include screening the plant, plant structure, or plant cell to determine if a double-stranded break (DSB) has occurred at or near the sequence targeted by the gRNA (crRNA and tracrRNA or the cr tracrRNA hybrid). For example, the PCR-digest assay described by Zhang et al. (supra) can be used to determine whether a DSB has occurred. Other useful methods include, without limitation, the T7 assay, the Surveyor assay, and southern blotting (if a restriction enzyme binding sequence is present at or near the predicted cleavage site).

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

Unless defined otherwise, all technical and scientific terms used have the same meaning as commonly understood by one of ordinary skill in the art to which this disclosure belongs. Although any methods and materials similar or equivalent to those described can also be used in the practice or testing of the present disclosure, the preferred methods and materials are now described.

Embodiments of the present disclosure will employ, unless otherwise indicated, techniques of separating, testing, and constructing materials, which are within the skill of the art. Such techniques are explained fully in the literature.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

1) A method for modifying the genomic material in a plant, comprising:
(a) providing a RNA-guided endonuclease and cell penetrating peptide (CPP) conjugate;
(b) providing one or more guide RNA (gRNA) and CPP conjugates;
(c) incubating mature pollen grains from a donor plant in a solution comprising the RNA-guided endonuclease and CPP conjugate and one or more guide RNA and CPP conjugates to form treated pollen grains; and
(d) pollinating a maternal plant with the treated pollen grains to produce a plant with modified genomic material.

2) The method of claim 1, wherein the RNA-guided endonuclease is a Cas9, a Cpf1, a C2c1, or a C2c2.

3) The method of claim 1, wherein the CPP comprises one of SEQ ID NOs. 1-16 or a modified variant thereof.

4) (canceled)

5) The method of claim 1, wherein the gRNA comprises crRNA and a tracrRNA, or a chimeric cr/tracrRNA hybrid.

6) The method of claim 1, wherein the donor plant is a monocotyledonous plant, food plant, or dicotyledonous plant.

7) The method of claim 1, wherein the donor plant is a wheat, maize, rice, orchid, onion, aloe, true lily, grass, Setaria, woody shrub, tree, palm tree, bamboo, pineapple, sugar cane, tomato, cassava, soybean, tobacco, potato, Arabidopsis, rose, pansy, sunflower, grape, strawberry, squash, bean, pea, or peanut.

8) The method of claim 1, wherein the donor plant is tobacco or maize.

9-16) (canceled)

17) The method of claim 1, wherein the solution further comprises a donor DNA sequence and CPP conjugate.

18) The method of claim 1, further comprising, before step (a), incubating a RNA-guided endonuclease and cell penetrating peptide (CPP) for at least 30 minutes to form a RNA-guided endonuclease and CPP conjugate.

19-20) (canceled)

21) The method of claim 18, further comprising, before steps (a) or (b), incubating one or more guide RNA (gRNA) and cell penetrating peptide (CPP) for at least 30 minutes to form one or more gRNA and CPP conjugates.

22-23) (canceled)

24) The method of claim 1, further comprising electroporating, sonicating, or both, the mature pollen grains in solution during step (c), after step (c), or both.

25) The method of claim 1, further comprising storing the treated pollen after step (c).

26) The method of claim 1, further comprising screening the treated pollen for double strand breaks after step (c).

27) The method of claim 1, further comprising screening the genotype of the plant with modified genomic material, phenotype of the plant with modified genomic material, or both, after step (d).

28) A kit, comprising:
one or more mature pollen grains;
a RNA-guided endonuclease;
one or more gRNA; and
a CPP.

29) The kit of claim 28 wherein the RNA-guided endonuclease is a Cas9, a Cpf1, a C2c1, or a C2c2.

30) The kit of claim 28, wherein the CPP comprises one of SEQ ID NOs. 1-16 or a modified variant thereof.

31) (canceled)

32) The kit of any claim 28, wherein the gRNA comprises crRNA and a tracrRNA, or a chimeric cr/tracrRNA hybrid.

33) The kit of claim 28, wherein one or more mature pollen grains are from a monocotyledonous plant, food plant, or dicotyledonous plant.

34) (canceled)

35) The kit of claim 28, wherein one or more mature pollen grains are tobacco or maize.

36-39) (canceled)
